(19) United States
(12) Patent Application Publication
(10) Pub. No.: US 2025/0090604 A1
(43) Pub. Date: Mar. 20, 2025

(54) STRAIN OF PEDIOCOCCUS ACIDILACTICI WITH FUNCTION OF TREATING ULCERATIVE COLITIS AND APPLICATION THEREOF

(71) Applicant: Shenzhen Porshealth Bioengineering Co., Ltd., Shenzhen (CN)

(72) Inventors: Yanyi Zheng, Shenzhen (CN); Silu Zhang, Shenzhen (CN); Guoxun Xiao, Shenzhen (CN); Zhizhu Zhang, Shenzhen (CN); Caijuan Lan, Shenzhen (CN)

(21) Appl. No.: 18/964,445

(22) Filed: Dec. 1, 2024

(30) Foreign Application Priority Data
Dec. 19, 2023 (CN) 202311762314.0

(51) Int. Cl. A61K 35/744; A61P 1/00; A61P 1/04; A61P 29/00

(52) U.S. Cl. CPC A61K 35/744; A61P 1/00; A61P 1/04; A61P 29/00

(57) ABSTRACT

A strain of Pediococcus acidilactici with a function of treating ulcerative colitis and its application are disclosed. The disclosure provides a strain of Pediococcus acidilactici GOLDGUT-PA0755, which can relieve ulcerative colitis, inhibit inflammation and repair intestinal barriers, specifically in: relieving the weight loss, colon length ratio reduction and DAI score increase caused by DSS of ulcerative colitis model mice, reducing the rise of the serum inflammatory factor level and the rise of the ratio of the inflammatory factor to the anti-inflammatory factor caused by DSS of ulcerative colitis model mice, recovering the injury of colonic mucosa caused by DSS of ulcerative colitis model mice, reducing the decrease of anti-inflammatory factors and the increase of the ratio of the inflammatory factor to the anti-inflammatory factor caused by the DSS of PBMC cells, and restoring the content of the tight junctional protein ZO-1 in inflammation injury model of monolayer epithelium.

### SEQUENCE LISTING

The sequence listing is submitted as a XML file filed via EFS-Web, with a file name of “Sequence_Listing.XML”, a creation date of Nov. 27, 2024, and a size of 5,155 bytes. The sequence Listing filed via EFS-Web is a part of the specification and is incorporated in its entirety by reference herein.

### TECHNICAL FIELD

The disclosure relates to a strain of Pediococcus acidilactici with a function of treating ulcerative colitis and its application, belonging to the technical field of biological medicines.

### BACKGROUND ART

Ulcerative colitis (UC) is a type of inflammatory bowel disease. The clinical manifestations of ulcerative colitis patients mainly include abdominal pain, diarrhea, hematochezia, etc. Ulcerative colitis mainly involves the rectum and colon, and is often a chronic course of recurrent attacks. The etiology of ulcerative colitis is currently unknown, but poor lifestyle, dysbacteriosis of the intestinal tract and the like may be its risk factors.

At present, ulcerative colitis has no thorough treatment means, and anti-inflammatory treatment or symptom alleviation is clinically carried out mainly aiming at inflammation caused by ulcerative colitis. Colectomy is needed if the condition is severe, there is no improvement after treatment, or complications such as colorectal cancer occur.

At present, common medicines for anti-inflammatory treatment or symptom alleviation aiming at inflammation caused by ulcerative colitis mainly include hormone medicines such as prednisone and the like, 5-aminosalicylic acid derivatives such as sulfasalazine and the like, and biological preparations such as infliximab and the like. However, long-term use of the above drugs may cause immune tolerance or liver injury. Thus, there is an urgent need to find more effective therapeutic agents for ulcerative colitis that are less prone to immune tolerance or liver injury.

### SUMMARY

In order to solve the above problems, the disclosure provides Pediococcus acidilactici GOLDGUT-PA0755, wherein the Pediococcus acidilactici GOLDGUT-PA0755 was deposited in China General Microbiological Culture Collection Center (CGMCC) with a deposit number of No. 28631 on Oct. 16, 2023.

The Pediococcus acidilactici GOLDGUT-PA0755 was derived from fresh fecal samples of healthy people in Shenzhen region. After sequencing and analysis, the 16S rDNA sequence of Pediococcus acidilactici GOLDGUT-PA0755 was shown in SEQ ID NO. 3. The sequence obtained by sequencing was subjected to nucleic acid sequence alignment in GeneBank, and the result showed that the strain was Pediococcus acidilactici and was named as Pediococcus acidilactici GOLDGUT-PA0755.

The disclosure further provides application of the Pediococcus acidilactici GOLDGUT-PA0755 in preparing a drug, wherein the drugs have any one of the following functions:
(a) preventing and/or treating ulcerative colitis;
(b) inhibiting inflammation; and/or
(c) repairing intestinal barrier.

In one embodiment of the disclosure, the components of the drug include the Pediococcus acidilactici GOLDGUT-PA0755, a drug carrier and/or a drug adjuvant.

In one embodiment of the disclosure, the drug carrier includes a microcapsule, a microsphere, a nanoparticle and/or a liposome.

In one embodiment of the disclosure, the drug adjuvant includes an excipient and/or an additive.

In one embodiment of the disclosure, the excipient includes a solvent, a propellant, a solubilizer, a cosolvent, an emulsifier, a colorant, an absorbent, a diluent, a flocculant, a deflocculant, a filter aid, and/or a release retardant.

In one embodiment of the disclosure, the additive includes microcrystalline cellulose, hydroxypropyl methylcellulose, and/or refined lecithin.

In one embodiment of the disclosure, the dosage form of the drug includes a powder, a granule, a capsule, a tablet, a pill or an oral liquid.

In one embodiment of the present disclosure, a viable count of the aforementioned Pediococcus acidilactici GOLDGUT-PA0755 in the drug is not less than 1×10⁶ CFU/mL or 1×10⁶ CFU/g.

The disclosure also provides a product, and the components of the product include the Pediococcus acidilactici GOLDGUT-PA0755.

In one embodiment of the disclosure, the product is a food or a drug.

In one embodiment of the disclosure, the components of the food further include a functional food or a food additive.

In one embodiment of the disclosure, the food additive includes an antioxidant, a bleach, a colorant, a color fixative, an enzyme preparation, a flavoring enhancer, a preservative, and/or a sweetener.

In one embodiment of the disclosure, the functional food includes functional beverage, special medical food or health care product.

In one embodiment of the disclosure, the drug has any of functions indicated below:
(a) preventing and/or treating ulcerative colitis;
(b) inhibiting inflammation; and/or
(c) repairing intestinal barrier.

In one embodiment of the disclosure, the components of the drug further include a drug carrier and/or a drug adjuvant.

In one embodiment of the disclosure, the drug carrier includes a microcapsule, a microsphere, a nanoparticle and/or a liposome.

In one embodiment of the disclosure, the drug adjuvant includes an excipient and/or an additive.

In one embodiment of the disclosure, the excipient includes a solvent, a propellant, a solubilizer, a cosolvent, an emulsifier, a colorant, an absorbent, a diluent, a flocculant, a deflocculant, a filter aid, and/or a release retardant.

In one embodiment of the disclosure, the additive includes microcrystalline cellulose, hydroxypropyl methylcellulose, and/or refined lecithin.

In one embodiment of the disclosure, the dosage form of the drug includes a powder, a granule, a capsule, a tablet, a pill or an oral liquid.

In one embodiment of the present disclosure, a viable count of the aforementioned Pediococcus acidilactici GOLDGUT-PA0755 in the drug is not less than 1×10⁶ CFU/mL or 1×10⁶ CFU/g.

The technical scheme of the disclosure has the following advantages:
the disclosure provides a Pediococcus acidilactici GOLDGUT-PA0755, and researches show that the Pediococcus acidilactici GOLDGUT-PA0755 can effectively relieve ulcerative colitis, inhibit inflammation and repair intestinal barriers, and is specifically characterized in that:
firstly, the Pediococcus acidilactici GOLDGUT-PA0755 can remarkably reduce the weight loss caused by DSS of ulcerative colitis model mice;
secondly, the Pediococcus acidilactici GOLDGUT-PA0755 can remarkably relieve the colon length ratio reduction caused by DSS of ulcerative colitis model mice;
thirdly, the Pediococcus acidilactici GOLDGUT-PA0755 can remarkably relieve the DAI score increase caused by DSS of ulcerative colitis model mice;
fourth, the Pediococcus acidilactici GOLDGUT-PA0755 can remarkably reduce the rise of the serum inflammatory factor level and the rise of the ratio of the inflammatory factor to the anti-inflammatory factor caused by DSS of ulcerative colitis model mice, and reduce the inflammatory level;
fifth, the Pediococcus acidilactici GOLDGUT-PA0755 can remarkably recover the injury of colonic mucosa caused by DSS of ulcerative colitis model mice, maintain the integrity of colon and lighten the level of colonitis;
sixth, the Pediococcus acidilactici GOLDGUT-PA0755 can remarkably reduce the decrease of anti-inflammatory factors and the increase of the ratio of the inflammatory factor to the anti-inflammatory factor caused by the DSS of PBMC cells, and reduce the inflammatory level;
seventh, the Pediococcus acidilactici GOLDGUT-PA0755 can obviously restore the content of the tight junctional protein ZO-1 in inflammation injury model of monolayer epithelium, and repair the intestinal barrier.

Therefore, the Pediococcus acidilactici GOLDGUT-PA0755 has great application prospects in preparing drugs for preventing and/or treating inflammatory bowel diseases and drugs for preventing and/or treating inflammation.

In addition, the Pediococcus acidilactici GOLDGUT-PA0755 has strong resistance to gastrointestinal fluid and air, which is beneficial to subsequent product development.

In addition, the Pediococcus acidilactici GOLDGUT-PA0755 can be used in a strain list of food, has the advantages of high safety and difficulty in generating immune tolerance or liver injury, and can not cause complications and side effects of patients after long-term use.

### Deposit of Biological Material

A strain of Pediococcus acidilactici GOLDGUT-PA0755, having a taxonomic designation of Pediococcus acidilactici, was deposited on 16 Oct. 2013 at the China General Microbiological Culture Collection Center, having a deposit number of CGMCC No. 28631 and a deposit address of No. 3, Courtyard 1, West Beichen Road, Chaoyang District, Beijing.

### BRIEF DESCRIPTION OF THE DRAWINGS

FIG. 1: effect of Pediococcus acidilactici GOLDGUT-PA0755 on body weight of dextran sulfate sodium salt (DSS) induced ulcerative colitis (UC) model mouse.

FIG. 2: body weight change of Pediococcus acidilactici GOLDGUT-PA0755 intervened group of UC mice on the tenth day of the experiment.

FIG. 3: effect of Pediococcus acidilactici GOLDGUT-PA0755 on colon length of DSS-induced UC mouse.

FIG. 4: effect of Pediococcus acidilactici GOLDGUT-PA0755 on DAI score of DSS-induced UC mouse.

FIG. 5: DAI score of Pediococcus acidilactici GOLDGUT-PA0755-interfered group of UC mice on the tenth day of the experiment.

FIG. 6: effect of Pediococcus acidilactici GOLDGUT-PA0755 on serum IL-6 (interleukin-6) content of DSS-induced UC mice.

FIG. 7: effect of Pediococcus acidilactici GOLDGUT-PA0755 on the serum IL-6/IL-10 (interleukin-10) ratio of DSS-induced UC mice.

FIG. 8: influence of Pediococcus acidilactici GOLDGUT-PA0755 on pathological sections of the colon of DSS-induced of UC mice.

FIG. 9: influence of Pediococcus acidilactici GOLDGUT-PA0755 on the expression level of IL-10 in the supernatant of LPS-induced peripheral blood mononuclear cells (PBMC).

FIG. 10: effect of Pediococcus acidilactici GOLDGUT-PA0755 on the IL-6/IL-10 ratio in the supernatant of LPS-induced peripheral blood mononuclear cells.

FIG. 11: effect of Pediococcus acidilactici GOLDGUT-PA0755 on TNF-α (tumor necrosis factor-α)/IL-10 ratio in the supernatant of LPS-induced peripheral blood mononuclear cells.

FIG. 12: effect of Pediococcus acidilactici GOLDGUT-PA0755 on the transepithelial electrical resistance (TEER) of LPS-induced monolayer fused epithelial injury model.

FIG. 13: effect of Pediococcus acidilactici GOLDGUT-PA0755 on the expression level of ZO-1 protein on the cell membrane surface of LPS-induced monolayer fused epithelial injury model.

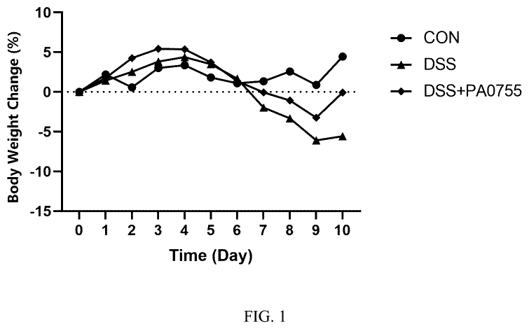

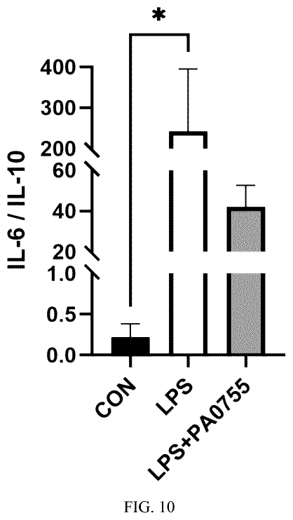

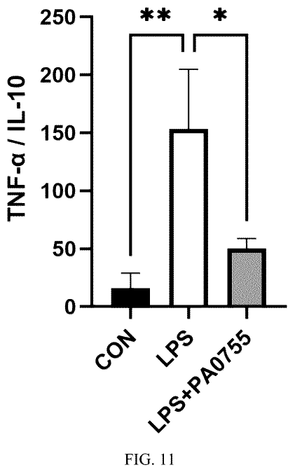

In FIG. 1-FIG. 13, *, p<0.05; **, p<0.01; ***, p<0.001; ****, p<0.0001.

### DETAILED DESCRIPTION OF THE EMBODIMENTS

The following embodiments are provided to better understand the present disclosure, are not limited to the best embodiments, and do not limit the content and scope of protection of the present disclosure. Any product that is the same as or similar to the present disclosure and is obtained by combining the present disclosure with other features of the prior art with the motivation of the present disclosure, falls within the scope of protection of the present disclosure.

The following embodiments relate to the following culture media:

MRS solid medium: peptone 10 g/L, beef extract powder 5 g/L, yeast extract powder 4 g/L, glucose 20 g/L, sodium acetate 5 g/L, dipotassium hydrogen phosphate 2 g/L, triammonium citrate 2 g/L, magnesium sulfate 0.2 g/L, manganese sulfate 0.05 g/L, Tween 80 1 g/L, agar 15 g/L, cysteine ammonia salt 0.5 g/L, pH 6.8.

MRS liquid medium: peptone 10 g/L, beef extract powder 5 g/L, yeast extract powder 4 g/L, glucose 20 g/L, sodium acetate 5 g/L, dipotassium hydrogen phosphate 2 g/L, triammonium citrate 2 g/L, magnesium sulfate 0.2 g/L, manganese sulfate 0.05 g/L, Tween 80 1 g/L, cysteine ammonia salt 0.5 g/L, pH 6.8.

BHI liquid medium: 37 g of BHI broth powder was weighed, added to 1000 mL of ultrapure water, and 1 g of L-cysteine hydrochloride and 0.01 g of hemin were added. After complete dissolution, sterilization was performed at 121° C. for 15 min. When the medium was cooled to 60° C., 1 mg of vitamin K1 was added and mixed well to obtain BHI liquid medium.

BHI solid medium: 37 g of BHI broth powder was weighed, added to 1000 mL of ultrapure water, and 1 g of L-cysteine hydrochloride, 0.01 g of hemin, and 2% (m/v, g/100 mL) of agar powder were added. After complete dissolution, sterilization was performed at 121° C. for 15 min. When the medium was cooled to 60° C., 1 mg of vitamin K1 was added and mixed well, then the mixture was poured into a plate, and stood in an anaerobic workbench for 16 h to obtain BHI solid medium.

The detection methods involved in the following embodiments are as follows:

Detection method of viable bacteria: the national standard “GB 4789.35-2016 national food safety standard, food microbiology detection, lactic acid bacteria detection” was adopted.

Detection method of colon length: after euthanasia of the mice, the entire colon (from the end of the cecum to the anus) was taken and the length was measured.

Detection method of disease activity index (DAI): the DAI scoring system included three aspects, weight change, hematochezia condition and feces traits (specific scoring criteria were shown in table 1). During the modeling period, the body weight, the hematochezia condition and the feces traits of the mice were examined daily, and the score was given according to table 1, with the DAI score being the total score of three results divided by 3, i.e., DAI score=(weight change score+hematochezia score+feces trait score)/3. Fecal occult blood conditions were determined using fecal occult blood (OB) reagent (pilami semi-quantitative assay) (purchased from Zhuhai Besso company). If there was reddish-brown or bright red blood visible to the naked eyes in the feces, it was bloody feces with naked eyes. Feces traits were classified into three classes: normal, loose and thin feces, and normal feces of mice were shaped into particles; if the feces had increased viscosity and were easy to scatter, but did not adhere to anus, the feces were loose; if the feces were not formed or were watery and adhered to the anus, they were thin feces.

TABLE 1
Disease activity index scoring criteria

| Weight | Occult blood/Bloody feces | | |
|---|---|---|---|
| loss (%) | with naked eyes | Feces traits | Score |
| 0 | Negative in occult blood | Normal state | 0 |
| 1~5 | Negative in occult blood | Loose | 1 |
| 6~10 | Negative in occult blood | Loose | 2 |
| 11~15 | Negative in occult blood | Thin feces | 3 |
| >15 | Blood feces with naked | Thin feces | 4 |
| | eyes | | |

### Experimental Example 1: Acquisition of Pediococcus acidilactici GOLDGUT-PA0755

The experimental example provides an acquisition process of the Pediococcus acidilactici GOLDGUT-PA0755, and the specific process is as follows:

Fresh feces of healthy people from Shenzhen region were taken as a sample, 0.5 mL of the sample was pipetted, added into 5 mL of BHI liquid medium, and cultured for 24 h at 37° C. in an anaerobic workstation (Electrok AW500 TG) for enrichment to obtain an enriched sample; the 0.5 mL of the enriched sample was pipetted into 4.5 mL of sterile physiological saline to obtain a 10⁻¹ diluent and then 0.5 mL of the 10⁻¹ diluent was pipetted into 4.5 mL of physiological saline to obtain a 10⁻² diluent, then this procedure was followed to obtain 10⁻³, 10⁻⁴, 10⁻⁵ and 10⁻⁶ diluents in turn; 100 μL of the gradient diluent was pipetted and coated on the MRS solid medium with one plate for each gradient of 10⁻⁴, 10⁻⁵ and 10⁻⁶, the same was incubated at 37° C. for 48 h in an anaerobic workstation (Electrok AW500 TG) to obtain colonies; the colonies with typical characteristics of Pediococcus acidilactici on the MRS solid medium were selected according to the shape, size, edge, transparency and the like of the colonies, then the selected colonies were picked out with the inoculation ring and scribed on the MRS solid medium, and cultured at 37° C. for 48 h in an anaerobic workstation (Electrotek AW500TG) to obtain purified single colonies; the purified single colonies were selected and inoculated into 5 mL MRS liquid medium respectively, and cultured for 24 h at 37° C. in an anaerobic workstation (Electrotek AW500TG) to obtain bacterial liquids; after numbering each strain corresponding to each bacterial liquid, Gram staining, strain identification, physiological and biochemical experiments and genome identification analysis were performed by referring to the steps described in textbook “Microbiology” (edited by Shen Ping and Chen Xiangdong), hemolytic activity test was performed by referring to the steps described in 3.7 of “Technical guidelines for safety inspection and evaluation of strains for health food raw materials” (2020 version), and a strain with typical characteristics of Pediococcus acidilactici was selected to obtain a strain of GOLDGUT-PA0755.

Wherein, the Gram staining process is as follows:

A single colony of GOLDGUT-PA0755 was picked out and subjected to bacterial smear, then the bacterial smear was dried, heated and fixed; after crystal violet was dripped for dyeing for 10 s, the bacterial smear was washed with water and spin-dried; after iodine solution was dripped for dyeing for 10 s, the bacterial smear was washed with water and spin-dried; after decolorizing solution was dripped for decolorizing for 10 s, the bacterial smear was washed with water and spin-dried; after sallow solution was dripped for counterstaining for 10 s, the bacterial smear was washed with water; after the bacterial smear was naturally dried, one drop of cedar oil was dripped into the bacteria coating position for oil-microscopic observation, and the observation results were that: GOLDGUT-PA0755 had a purple Gram stain and was a spherical Gram positive bacterium.

The strain identification process is as follows:

GOLDGUT-PA0755 was taken and its genome was extracted by a bacterial genome extraction kit, and using a 27F/1492R primer pair (27F: AGAGTTTGATCCGTCTCA, 1492R: TGTACGGY TACCTTGTTACTACGACTT, in 27F and 1492R, M and Y are degenerate bases, M=A or C, Y=C or T), the amplification was performed by using the extracted genome of GOLDGUT-PA0755 as a template to obtain 16S rRNA of GOLDGUT-PA0755 (16 SrDNA sequence of GOLDGUT-PA0755 is shown as SEQ ID NO. 3); the 16S rDNA of GOLDGUT-PA0755 was subjected to nucleic acid sequence alignment in GeneBank, and the result showed that the strain was Pediococcus acidilactici, named as Pediococcus acidilactici GOLDGUT-PA0755.

The physiological and biochemical experimental process is as follows:

A single colony of GOLDGUT-PA0755 was picked out with sterile cotton swab, inoculated into sterile saline, and a homogeneous bacterial suspension was prepared by using calibrated VITEK® 2 DensiCHEK™ Plus at a turbidity corresponding to McFarland turbidity of 3.0 and was submitted to physiological and biochemical tests in ANC cards within 30 min of preparation, which showed that GOLDGUT-PA0755 was able to ferment D-glucose, D-cellobiose, D-mannose, D-ribose, N-acetyl-D-glucosamine, and did not metabolize D-maltose, sucrose, arbutin, maltotriose, L-arabinose and D-xylose.

The genome identification analysis process is as follows:

GOLDGUT-PA0755 was taken and its DNA was extracted with SDS method, the quality of extracted DNA was detected by agarose electrophoresis, and DNA quantification was performed by Qubit® 2.0; the genome was sequenced by using Nanopore PromethION platform and Illumina NovaSeq platform, and the sequencing strategy was a 10 Kb library with a sequencing depth of more than or equal to 100×. The sequencing results were: the strain GOLDGUT-PA0755 contained a circular genome with the size of 1.85 Mbp, and also contained one circular plasmid with the size of 39.54 kbp. The amino acid sequence encoded by the genome of strain GOLDGUT-PA075 was aligned with VFDB database, and the consistency of potential virulence factor sequences obtained through analysis was below 85%, and many of them did not have clear functions. The amino acid sequence encoded by the genome of strain GOLDGUT-PA075 was aligned with ARDB database to analyze potential resistance genes, and it was found that the consistency of all potential resistance gene sequences in the GOLDGUT-PA075 genome was below 55%.

### Experimental Example 2: Effect of Pediococcus acidilactici GOLDGUT-PA0755 on Ulcerative Colitis of DSS-Induced Mice

The experimental example provides an experiment for effect of Pediococcus acidilactici GOLDGUT-PA0755 on ulcerative colitis of DSS-induced mice, and the experimental process was as follows:

30 female mice (obtained from Guangdong Weitong Lihua Biotechnology Co., ltd., with weight of 19±2 g) of SPF grade C57BL/6 at 8 weeks of age were randomly divided into three groups of 10 animals each, and the three groups were: blank control group (CON), model control (DSS) group and GOLDGUT-PA0755-interfered group (DSS+GOLDGUT-PA0755). The experimental animals were raised for 9 days, wherein drinking water of mice in the blank control group was sterile water, and the blank control group was orally administered 200 μL of 0.9% (m/v, g/100 mL) physiological saline by gavage daily; the model control group was given 2.5% (m/v, g/100 mL) DSS aqueous solution instead of drinking water on days 1-7 of the experiment, and was orally administered 200 μL of 0.9% (m/v, g/100 mL) physiological saline by gavage daily; the GOLDGUT-PA0755-interfered group drank 2.5% (m/v, g/100 mL) DSS aqueous solution on days 1-7 of the experiment, and was orally administered 200 μL of Pediococcus acidilactici GOLDGUT-PA075 bacterial solution (dissolved in physiological saline) with the concentration of 2×10⁹ CFU/mL by gavage daily. On days 8-9 of the experiment, the drinking water of all groups of mice was replaced with sterile water and the gavage was stopped. On the 10th day as the endpoint of the experiment, after euthanizing all mice, inflammation evaluation was conducted according to the evaluation criteria.

The evaluation criteria for the inflammation evaluation are as follows:

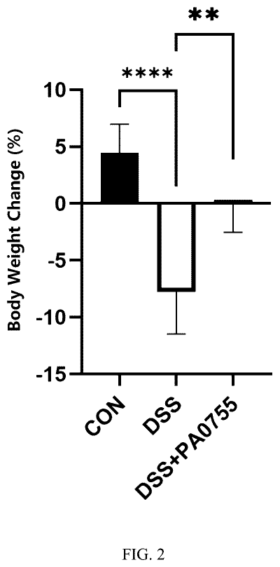

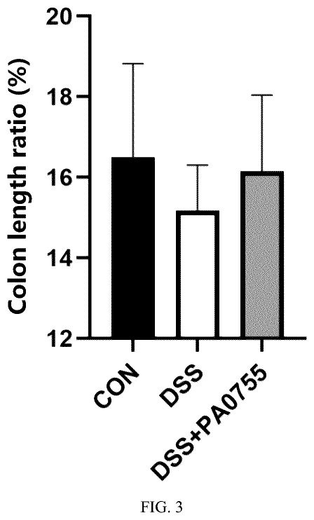

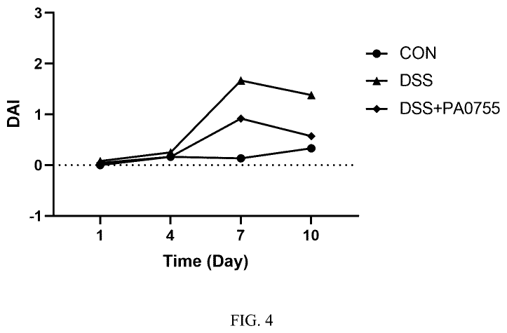

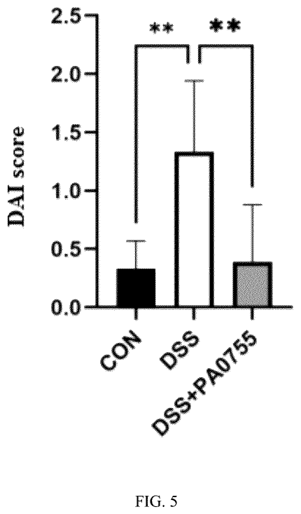

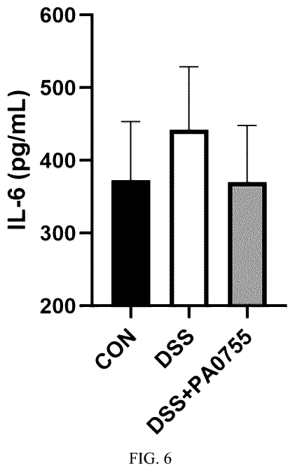

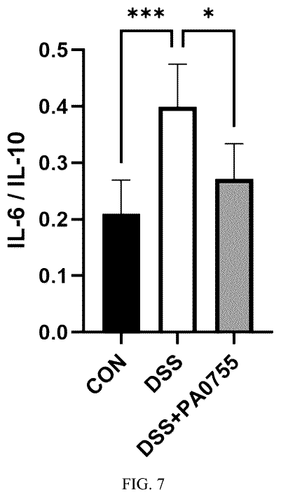

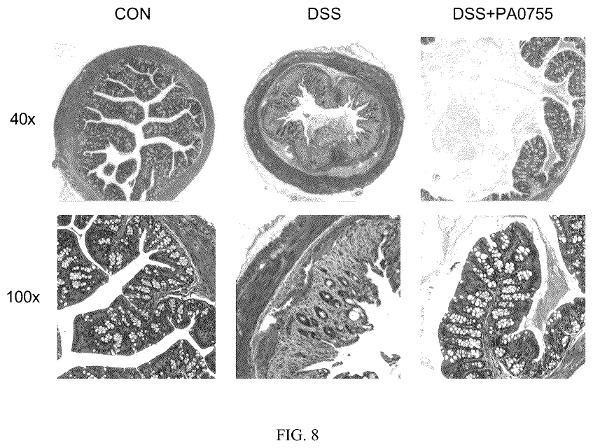

from the first day of the experiment to the end of the experiment, the weights of all groups of mice were recorded daily and analyzed for daily weight changes, and the statistical results were shown in FIGS. 1 and 2. After euthanizing mice at the experimental endpoint, the colons of all groups of mice were taken down, the length of the colon and the total length of the intestinal tract were measured, the proportion of the colon to the total length of the intestinal tract was calculated, and the statistical result was shown in FIG. 3. On days 1, 4, 7, 10 of the experiment, the faeces of all groups of mice were collected and subjected to DAI scoring and the scoring was analyzed for changes, and the scoring results were shown in FIGS. 4 and 5. After euthanizing mice at the endpoint of the experiment, blood of all groups of mice was rapidly collected and serum was separated, and the content of cytokines in the serum was detected using an inflammatory factor flow assay kit (purchased from Biolegend corporation), and the ratio of inflammatory factors IL-6 and IL6/IL-10 was calculated, and the statistical results were shown in FIGS. 6 and 7. After euthanizing mice at the endpoint of the experiment and measuring the colon length, the colon segments of all groups of mice with the length of 0.5 cm was sheared off and fixed by 4% (m/v, g/100 mL) paraformaldehyde; after fixation, paraffin section preparation and H&E staining were performed to evaluate intestinal pathology results, which were shown in FIG. 8.

The experimental results are as follows:

as shown in FIGS. 1 and 2, on day 5 of DSS drinking of mice, body weight began to drop, while Pediococcus acidilactici GOLDGUT-PA0755 intragastric treatment alleviated the extent of body weight drop caused by DSS drinking. On day 9, the mice in the model control group and the GOLDGUT-PA0755-interfered group reached the lowest body weight point. When the experiment proceeded to day 10, the mice in each group had slightly increased body weight, with the GOLDGUT-PA0755-interfered group having more significant increase relative to the model control group. Compared with the starting point of the experiment, the weight of the mice in the blank control group was increased by about 4% compared with the initial value, the weight of the mice in the model control group was obviously reduced by about 8%, and after the gavage treatment by the Pediococcus acidilactici GOLDGUT-PA0755, the weight reduction trend of the mice in the GOLDGUT-PA0755-interfered group was obviously relieved compared with the model control group, which had almost no change compared with the initial value. It was demonstrated that Pediococcus acidilactici GOLDGUT-PA0755 significantly reduced the weight loss trend caused by DSS.

As shown in FIG. 3, after DSS drinking of mice, the colon length ratio decreased (from 16.5% to 15.2%, and a decrease of 1.3% compared to the blank control group). After the gavage treatment by the Pediococcus acidilactici GOLDGUT-PA0755, the reduction of the colon length ratio of the mice was relieved (from 15.2% to 16.2%, and an increase of 1% compared to the model control group), and the colon length ratio of the mice in the GOLDGUT-PA0755-interfered group was not greatly different from that of the mice in the blank control group.

As shown in FIGS. 4 and 5, the DAI score of the mice in the blank group was kept low (0 to 0.3) throughout the experimental time. While the DAI scores of the other two groups of mice were increased, but the DAI score of the model control group of mice were higher than that of the GOLDGUT-PA0755-interfered group of mice. At the endpoint of the experiment, the DAI score of GOLDGUT-PA0755-interfered group of mice was significantly reduced compared to that of the model control group, wherein the score of the model control group reached 1.4, while the score of the GOLDGUT-PA0755-interfered group was only 0.4.

As shown in FIGS. 6 and 7, compared to the control group, in the serum of the model control group, the inflammatory factor IL-6 level was increased (by about 20%), while after the gavage treatment with Pediococcus acidilactici GOLDGUT-PA0755, the IL-6 level showed a downward trend (by about 20%), which was substantially consistent with the blank control group. Analysis of the ratio of inflammatory factor IL-6 to anti-inflammatory factor IL-10 revealed that the ratio of inflammatory factor to anti-inflammatory factor was significantly increased (about 1-fold increase) in mice in the DSS-induced model control group compared to the blank control group, whereas the ratio of mice in the GOLDGUT-PA0755-interfered group showed a significant decrease (about 30% decrease) compared to the DSS-induced model control group. The above results show that the Pediococcus acidilactici GOLDGUT-PA0755 could obviously reduce the overall inflammation degree in mice, and played a role in inhibiting inflammation.

As shown in FIG. 8, the colon tissue of the mice in the blank group was normal, the mucosal epithelial cells were intact, the crypts were normal, the glands were orderly arranged, and there was substantially no infiltration of immune cells. However, DSS caused severe injury to the colonic mucosa, and the colonic mucosa festered, so that the ordered glandular arrangement was not observed basically, and the immune cell infiltration phenomenon was observed obviously. Compared with the model control group, the mice in GOLDGUT-PA0755-interfered group had complete colon epithelium with only a few fester phenomena, glandular arrangement can be observed, and immune cell infiltration was obviously reduced. The above results demonstrate that the intervention of Pediococcus acidilactici GOLDGUT-PA0755 maintained the integrity of the colon, which reduced the level of colonic inflammation.

Based on the above results, the Pediococcus acidilactici GOLDGUT-PA0755 can reduce the inflammation level of mice in various aspects, maintain the intestinal structural integrity of the mice, and obviously reduce the ulcerative colitis symptoms caused by DSS.

The result shows that the interference of the Pediococcus acidilactici GOLDGUT-PA0755 has the effect of treating ulcerative colitis.

### Experimental Example 3: Effect of Pediococcus acidilactici GOLDGUT-PA0755 on Expression of Inflammatory Factors in LPS-Induced PBMC Cells

The experimental example provides an experiment for effect of Pediococcus acidilactici GOLDGUT-PA0755 on expression of inflammatory factors in LPS-induced PBMC cells, and the experimental process is as follows:

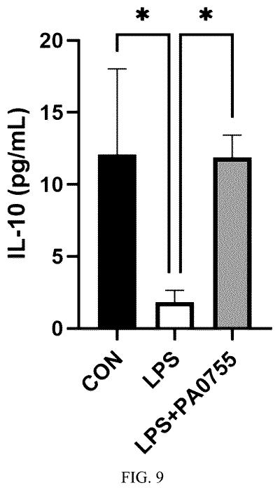

after removed from the liquid nitrogen tank, the PBMC cells (purchased from Shanghai Australian corporation) were shaken in a water bath at 37° C. for 3 min for unfreezing to obtain unfrozen PBMC cells; after resuspension of the unfrozen PBMC cells with 5 mL of PBS buffer pre-warmed to 37° C., 400×g centrifugation was performed for 10 min, the supernatant was discarded to obtain a precipitate A; the precipitate A was resuspended in 5 mL of cell medium (90% RPMI 1640 medium+10% fetal bovine serum+50 μg/mL double antibody, % referred to the volume ratio, RPMI 1640 medium and double antibody were purchased from Gibco company, fetal bovine serum was purchased from Solarbio company), and subjected to cell counting and viability detection to obtain a resuspension A; 8 mL of cell culture medium was added to resuspension A at 5% (v/v) CO₂ and was cultured for 6 h at 37° C. to obtain a culture solution; the culture solution was subjected to 400×g centrifugation for 10 min, the supernatant was discarded, and cell counting was performed to obtain a precipitate B; the precipitate B was resuspended to the concentration of 1×10⁶ cells/mL with cell culture medium to obtain a resuspension B; the suspension B was added to the 96-well plate at an addition amount of 100 μL per well, then lipopolysaccharide (LPS, purchased from Sigma-Aldrich Co.) was added to the 96-well plate at an addition amount of 1 μg/mL, and 1×10⁷ CFU/mL of the bacterial liquid of Pediococcus acidilactici GOLDGUT-PA0755 (with solvent of physiological saline) was added to the 96-well plate at an addition amount of 10 μL per well, and finally, anaerobic incubation was performed on the 96-well plate at 37° C. for 2 h; after the incubation, the 96-well plate was subjected to 500×g centrifugation for 5 min (at this time PBMC cells were collected to the bottom of the 96-well plate and Pediococcus acidilactici GOLDGUT-PA0755 was suspended in the supernatant), the supernatant was discarded to remove Pediococcus acidilactici GOLDGUT-PA0755 viable bacteria from the 96-well plate, then PBMC cells from 96-well plates were resuspended in PBS buffer containing 10% (m/v, g/100 mL) of double antibody, and the 96-well plate was subjected to 500×g centrifugation for 5 min again, the supernatant was discarded to wash out residual Pediococcus acidilactici GOLDGUT-PA0755 viable bacteria from the 96-well plate, and finally fresh cell culture medium was added to the 96-well plate at an addition amount of 125 μL per well and cultured at 5% (v/v) CO₂ at 37° C. for 22 h; after the completion of the culture, the interacted supernatant was collected by centrifugation, and the cytokine content in the supernatant was detected using a flow type multi-factor detection kit (purchased from Biolegend), as shown in FIGS. 9 to 11.

The experimental results are as follows:

as shown in FIGS. 9-11, induction of LPS could reduce the content of IL-10 in cell supernatant and increase the ratios of IL-6/IL-10 and TNF-alpha/IL-10, and the results showed that LPS could stimulate cells to produce inflammatory response. When the cells interacted with Pediococcus acidilactici GOLDGUT-PA0755, the content of IL-10 was significantly increased (about 5.5-fold increase), and the ratios of IL-6/IL-10 and TNF-alpha/IL-10 were significantly decreased (about 80% and 70% decrease, respectively). The results show that the Pediococcus acidilactici GOLDGUT-PA0755 has the function of inhibiting inflammation.

### Experimental Example 4: Effect of Pediococcus acidilactici GOLDGUT-PA0755 on LPS-Induced Monolayer Fused Epithelial Injury Model

The experimental example provides an experiment for effect of Pediococcus acidilactici GOLDGUT-PA0755 on LPS-induced monolayer fused epithelial injury model, and the experimental process was as follows:

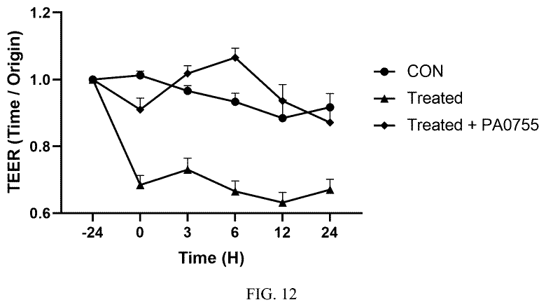

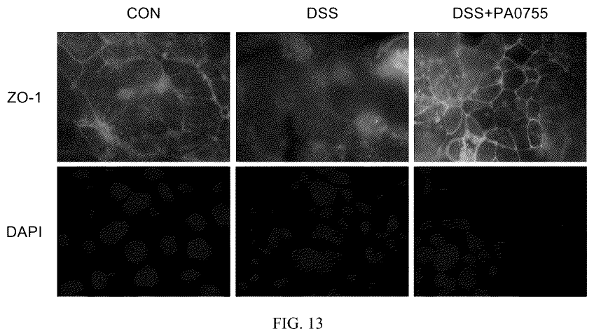

CACO-2 cells (purchased from Shanghai cell institute of China Academy of Sciences) were taken out from a liquid nitrogen tank and unfrozen in a water bath at 37° C. for 3 min to obtain unfrozen CACO-2 cells; CACO-2 cells were washed with PBS buffer, added with 8 mL of cell culture medium (the cell culture medium was a DMEM medium containing 10% fetal bovine serum, % referred to volume ratio, the DMEM medium was available from Gibco corporation, fetal bovine serum was available from Solarbio corporation), which was cultured at 5% (v % v) CO₂ at 37° C. for 48 h to logarithmic phase to obtain CACO-2 cells in logarithmic phase; CACO-2 cells in logarithmic phase and HT29-MTX-E12 cells (purchased from ATCC) were inoculated into the upper chamber of Transwell chambers at a ratio of 9:1, with a total inoculation size being 2.5×10⁵ μL; and 100 μL and 600 μL of cell culture medium were added to the upper and lower chambers, respectively, which was cultured at 5% (v:v) CO₂ and 37° C. for 24 h; after the culture, the cell culture medium in the upper and lower chambers was replaced with new cell culture medium (100 μL for the upper chamber and 600 μL for the lower chamber), which was cultured at 5% (v/v) CO₂ and 37° C., and the transepithelial electrical resistance was detected using a cell resistance meter; after the resistance value stabilized, the cells were completely fused, and CACO-2/HT29-MTX-E12 monolayer fused epithelium was obtained; after obtaining the fused epithelium, the cell culture medium in the upper and lower chambers was replaced with a cell culture medium additionally added with 100 μg/mL LPS+40 ng/mL TNF-α+100 ng/ML IL-1β (100 μL for the upper chamber and 600 μL for the lower chamber), which was cultured at 5% (v/v) CO2 and 37° C. for 24 h to obtain a monolayer epithelial inflammatory injury model; after obtaining the monolayer epithelial inflammatory injury model, the cell culture medium in the upper and lower chambers was removed, the Transwell chambers were washed with PBS buffer, and new cell culture medium was added to the upper and lower chambers (100 μL for the upper chamber and 600 μL for the lower chamber), respectively; then a bacterial solution containing 1×10⁷ CFU/mL Pediococcus acidilactici GOLDGUT-PA0755 (the solvent was physiological saline) was added into the upper chamber at an addition amount of 10 μL per well, and the mixture was subjected to anaerobic incubation for 2 h at 37° C.; after the incubation, the cell culture medium in the upper and lower chambers was removed, the Transwell chambers were washed with PBS buffer, 100 μL and 600 μL of double-antibody-free DMEM high-sugar cell culture medium (purchased from Gibco Co.) was added into the upper chamber and the lower chamber respectively, and culturing was performed in a cell culture box at 37° C. and 5% (v/v) CO₂ for 22 h; during culturing, the transepithelial electrical resistance (TEER, in units of Ω*cm²) value of monolayer fusion epithelial was detected at culture hours of −24 (before molding), 0, 3, 6 and 12 using a cell resistance meter, and the detection result was shown in FIG. 12; after 22 hours of culture, the medium in the upper and lower chambers was removed, the Transwell chambers were washed with PBS buffer, 100 μL of 4% (M/V, g/100 ml) paraformaldehyde precooled to 4° C. was added to the upper chamber and the lower chamber, and fixed at 4° C. for 25 min; after 25 min of fixation, the Transwell chambers were washed with PBS buffer, 200 μL of PBS buffer containing 0.2% (m/v, g/100 mL) Triton X-100 was added to the upper and lower chambers, and left on ice for 10 min; after 10 min of standing, the Transwell chambers were washed with PBS buffer, 300 μL of PBS buffer containing 5% (m/v, g/100 mL) BSA was added to the upper and lower chambers, and blocked for 1 h at room temperature (25° C.); After blocking for 1 h, anti-ZO-1 antibodies (purchased from Abcam Company) were added to the upper and lower chambers according to the instructions, and incubation was performed at 4° C. for 16 h; after incubation for 16 h, the Transwell chambers were washed with PBS buffer, FITC-labeled secondary antibodies (purchased from Biyuntian Company) were added to the upper and lower chambers according to the instructions, and incubation was performed for 2 h at room temperature (25° C.) and protected from light; after incubation for 2 h, the Transwell chamber was washed with PBS buffer, the membrane of the Transwell chamber was carefully peeled off, a mounting medium containing DAPI (purchased from Biyuntian Company) was added, and the mixture was placed in a glass bottom petri dish for observation under a fluorescence microscope, and the observation results were shown in FIG. 13.

The experimental results are as follows:

as shown in FIG. 12, the transepithelial electrical resistance value of the monolayer fused epithelial injury model treated with IL-1β, TNFα and LPS showed a significant decrease trend compared to the untreated monolayer fused epithelial, whereas treatment with Pediococcus acidilactici GOLDGUT-PA0755 was able to slow down the decrease of the transepithelial electrical resistance value of the monolayer fused epithelial injury model. At the end of the experiment, the transepithelial electrical resistance of the monolayer fused epithelial injury model treated with IL-1β, TNFα and LPS was reduced by about 27% compared to the untreated monolayer fused epithelial, and after treatment with Pediococcus acidilactici GOLDGUT-PA0755, the transepithelial electrical resistance of the monolayer epithelial injury model treated with IL-1β, TNFα and LPS was significantly increased (by about 29%) and recovered to a level similar to that of the untreated single-layer fused epithelial.

As shown in FIG. 13, the fluorescence intensity of the tight junctional protein ZO-1 in the monolayer fused epithelial injury model treated with IL-1β, TNFα and LPS was significantly reduced compared to that of the untreated monolayer fused epithelial, whereas the tight junctional protein ZO-1 content in the monolayer epithelial inflammatory injury model treated with Pediococcus acidilactici GOLDGUT-PA0755 was significantly recovered.

The above results show that Pediococcus acidilactici GOLDGUT-PA0755 had the function of intestinal barrier repair.

It is apparent that the above examples are given by way of illustration only and are not limiting of the embodiments. Other variations or modifications of the above teachings will be apparent to those of ordinary skill in the art. It is not necessary here nor is it exhaustive of all embodiments. While still being apparent from variations or modifications that may be made by those skilled in the art are within the scope of the disclosure.

What is claimed is:

1. A strain of Pediococcus acidilactici, wherein the Pediococcus acidilactici was deposited in China General Microbiological Culture Collection Center (CGMCC) with a deposit number of CGMCC No. 28631.

2. An application of the Pediococcus acidilactici of claim 1 in preparing a drug having any one of functions indicated below:
(a) preventing and/or treating ulcerative colitis;
(b) inhibiting inflammation; and/or
(c) repairing intestinal barrier.

3. The application of claim 2, wherein components of the drug comprise the Pediococcus acidilactici GOLDGUT-PA0755, a drug carrier and/or a drug adjuvant.

4. The application of claim 3, wherein the drug carrier comprises a microcapsule, a microsphere, a nanoparticle and/or a liposome; the drug adjuvant comprises an excipient and/or an additive; the excipient comprises a solvent, a propellant, a solubilizer, a cosolvent, an emulsifier, a colorant, an absorbent, a diluent, a flocculant, a deflocculant, a filter aid, and/or a release retardant; the additive comprises microcrystalline cellulose, hydroxypropyl methylcellulose, and/or refined lecithin.

5. A product, wherein components of the product comprise the Pediococcus acidilactici of claim 1.

6. The product of claim 5, wherein the product is a food or a drug; components of the food further comprise a functional food or a food additive; components of the drug further comprise a drug carrier and/or a drug adjuvant.

7. The product of claim 6, wherein the food additive comprises an antioxidant, a bleach, a colorant, a color fixative, an enzyme preparation, a flavoring enhancer, a preservative, and/or a sweetener; the drug carrier comprises a microcapsule, a microsphere, a nanoparticle and/or a liposome; the drug adjuvant comprises an excipient and/or an additive; the excipient comprises a solvent, a propellant, a solubilizer, a cosolvent, an emulsifier, a colorant, an absorbent, a diluent, a flocculant, a deflocculant, a filter aid, and/or a release retardant; the additive comprises microcrystalline cellulose, hydroxypropyl methylcellulose, and/or refined lecithin.

8. The product of claim 6, wherein the drug has any of functions indicated below:
(a) preventing and/or treating ulcerative colitis;
(b) inhibiting inflammation; and/or
(c) repairing intestinal barrier.

9. The drug of claim 6, wherein a dosage form of the drug comprises a powder, a granule, a capsule, a tablet, a pill or an oral liquid.

10. The drug of claim 6, wherein a viable count of the aforementioned Pediococcus acidilactici GOLDGUT-PA0755 in the drug is not less than 1×10⁶ CFU/mL or 1×10⁶ CFU/g.


SEQUENCE LISTING (3 sequences)

SEQ ID NO: 1 - DNA, 19 bp, synthetic construct
  source 1..19: mol_type = other DNA
agagtttgatcmtggctca

SEQ ID NO: 2 - DNA, 24 bp, synthetic construct
  source 1..24: mol_type = other DNA
tgtacggytaccttgttacgactt

SEQ ID NO: 3 - DNA, 1423 bp, Pediococcus acidilactici
  source 1..1423: mol_type = other DNA
accccaccggctttgggtgttacaaactctcatggtgtgacgggcggtgtgtacaaggcccgggaacgtattcaccgcggcatgctgatccgcgattactagcgattccgacttcgtgtaggcgagttgcagcctacagtccgaactgagaatggttttaagagattagctaaacctcgcggtttcgcgactcgttgtaccatccattgtagcacgtgtgtagcccaggtcataaggggcatgatgatttgacgtcgtccccaccttcctccggtttgtcaccggcagtctcactagagtgcccaactgaatgctggcaactagtaataagggttgcgctcgttgcgggacttaacccaacatctcacgacacgagctgacgacaaccatgcaccacctgtcattctgtccccgaagggaacgcctaatctcttaggttggcagaagatgtcaagacctggtaaggttcttcgcgtagcttcgaattaaaccacatgctccaccgcttgtgcgggcccccgtcaattcttttgagtttcaaccttgcggtcgtactccccaggcggattacttaatgcgttagctgcagcactgaagggcggaaaccctccaacacttagtaatcatcgtttacggcatggactaccagggtatctaatcctgttcgctacccatgctttcgagcctcagcgtcagttacagaccagacagccgccttcgccactggtgttcttccatatatctacgcatttcaccgctacacatggagttccactatcctcttctgcactcaagtctcccagtttccaatgcacttcttcggttgagccgaaggctttcacattagacttaaaagaccgcctgcgctcgctttacgcccaataaatccggataacgcttgccacctacgtattaccgcggctgctggcacgtagttagccgtggctttctggttaaataccgtcactgggtgaacagttactctcacccacgttcttctttaacaacagagctttacgagccgaaacccttcttcactcacgcggcgttgctccatcagacttgcgtccattgtggaagattccctactgctgcctcccgtaggagtctgggccgtgtctcagtcccaatgtggccgattaccctctcaggtcggctacgcatcatcgccttggtgagccgttacctcaccaactagctaatgcgccgcgggtccatccagaagtgatagcagagccatcttttaaaagaaaaccaggcggttttctctgttatacggtattagcatctgtttccaggtgttatcccctgcttctgggcaggttacccacgtgttactcacccgtccgccactcacttcgtgttaaaatctcattcagtgcaagcacgtcataatcaattaacggaagttcgttcgacttgc